(19) United States
(12) Patent Application Publication
(10) Pub. No.: US 2011/0052503 A1
(43) Pub. Date: Mar. 3, 2011

(54) BIODEGRADABLE CONTRAST AGENTS

(75) Inventors: Torsten Almen, Lund (SE); Bjarne Brudeli, Lund (SE); Fred Kjellson, Lund (SE); Jo Klaveness, Lund (SE); Jian-Sheng Wang, Lund (SE)

(73) Assignee: IOPHARMA TECHNOLOGIES AB, Lund (SE)

(21) Appl. No.: 12/808,318

(22) PCT Filed: Dec. 22, 2008
(86) PCT No.: PCT/GB2008/004268

§ 371 Date: Nov. 8, 2010

(30) Foreign Application Priority Data
Dec. 21, 2007 (GB) 0725070.7

(51) Int. Cl. A61K 49/04; G21F 1/10

(52) U.S. Cl. 424/9.4; 252/478

(57) ABSTRACT

The present invention provides a radio-opaque composition comprising a cleavable, preferably enzymatically-cleavable, derivative of a physiologically tolerable organoiodine compound and a non-acrylic polymer wherein said derivative is incorporated in said non-acrylic polymer.

The present invention relates to biodegradable contrast media for use in biomaterials, particularly contrast media which are biologically compatible with their surroundings, so as to cause no negative influence on blood or other surrounding tissues. Additionally, this invention relates to methods for preparing polymers containing biodegradable contrast media. Moreover, this invention relates to radio-opaque objects and methods for rendering objects radio-opaque.

The ability to render objects radio-opaque is important in several fields. For example, in medicine it is important for medical devices to be seen in X-ray investigations during medical procedures and post-operative follow-ups. Metallic implants can be monitored easily due to the radio-opacity of metals.

In the case of devices which are not radio-opaque, they can be manufactured to comprise a radio-opaque material, e.g. a compound with the ability to absorb X-rays (often termed an X-ray contrast agent). This allows the placement of the medical device to be monitored, e.g. shortly after an operation to insert a prosthesis or over the subsequent years. In general, such radio-opaque materials are compounds of heavy metals. Where the medical device is manufactured from a polymer, the heavy metal compound is incorporated into the polymer as insoluble particles. Barium sulphate and zirconium dioxide are commonly used in this manner. Other methods include coating the surfaces of the object with gold/silver ions. Radio-opaque paints and inks with barium sulphate or silver powders physically trapped in the compositions have also been proposed. For non-medical applications, lead can be used, typically in plated form or compounded into ceramics.

There are several disadvantages with the current methods of rendering objects radio-opaque. In particular, medical devices treated with the current methods often have low bio-compatibility because of their radio-opaque fillers. Additives in polymeric implants are liable to diffuse into the surroundings and may cause inflammatory responses. This can in the end cause undesirable responses like necrosis, pain and expulsion of the object.

For example most medical stents are constructed from metal, and they are therefore visible via X-ray investigations. Even though such metal stents possess certain favourable characteristics, they also exhibit a number of significant disadvantages. The likelihood of restenosis, a biological process where smooth muscle cells and matrix proteins further occludes the blood vessels, increases. Other disadvantages with the current methods in the medical and the industrial fields include galvanic corrosion, undesirable changes in the physical, mechanical and electromagnetic properties of the devices, high economic cost and cumbersome processes for producing the devices. Recently, biocompatible and/or bioresorbable polymer stents made of polymers of glycolic and lactic acid have been proposed for use in medical stent systems. However, these materials suffer from the disadvantage that they are not radio-opaque.

For devices manufactured from polymers, it has been proposed to utilize a compound comprising an iodophenyl group linked to an acrylic group via an ester group (e.g. 2-methacryloyloxyethyl (2,3,5-triiodobenzoate), 2-methacryloyloxypropyl (2,3,5-triiodobenzoate), and 3-methacryloyloxypropyl-1,2-bis(2,3,5-triiodobenzoate) (see Davy et al. Polymer International 43: 143-154 (1997)), 2,5-diiodo-8-quinolyl methacrylate (see Vazquez et al. Biomaterials 20: 2047-2053 (1999)), and 4-iodophenyl methacrylate (see Kruft et al. J. Biomedical Materials Res. 28: 1259-1266 (1994)) as a monomer in the preparation of the polymer matrix. It is clear however that the resulting polymer will not only contain residual unreacted organoiodine monomer, but that exposure to physiological fluids will result in the release of organoiodine compounds with unclear physiological compatibility.

The potential release of contrast agent from the polymer matrix is particularly problematic when a biodegradable polymer is used. As the polymer degrades, so the incorporated radio-opaque material is released. Biodegradable polymers comprising radio-opaque compounds may be used in a variety of fields, in many of which it is undesirable to have potentially toxic contract agents being released. It would be useful for a wide variety of biodegradable polymers to be made radio-opaque for use in temporary medical devices.

For example biodegradable polymers can be used in temporary medical devices such as clips, sutures etc. which are intended to degrade after time, but nonetheless need their positioning monitored for a period after implant. As the biodegradable polymer degrades (for example inside the body in the case of a degradable suture) the contrast agent will be released and thus insoluble particles or material of unknown physiological compatibility will be released into the surrounding tissues. Similar problems are found for non-biodegradable polymers as contrast agent compounds will be released from within the device should it break and from the surface of the device due to it being in contact with bodily fluids.

Current methods therefore have the drawbacks that by their particulate nature and/or the fact that they are not homogenously distributed within polymers, the contrast agents reduce the mechanical strength of the polymer matrix. Moreover any release of the radio-opaque material from the device distributes highly abrasive particles and/or toxic material. This is particularly problematic in medical applications where the mechanical strength of the implant is important and/or it is intended to degrade in the body over time, for example the case of degradable sutures etc. There thus exists a need for materials which are radio-opaque, mechanically strong and, if degraded (whether by accidental failure of the device or intended degradation) release only physiologically tolerable substances.

We have now realized that these problems may be addressed by combining a non-acrylic polymer with a cleavable, preferably enzymatically-cleavable, derivative of a physiologically tolerable organoiodine compound.

Viewed from a first aspect, the present invention provides a radio-opaque composition comprising a cleavable, preferably enzymatically-cleavable, derivative of a physiologically tolerable organoiodine compound and a non-acrylic polymer wherein said derivative is incorporated in, e.g. dissolved in or present as a monomer residue in, said non-acrylic polymer.

From a further aspect the invention provides a radio-opaque composition comprising the product of polymerising a non-acrylic monomer containing a cleavable, preferably enzymatically-cleavable, derivative of a physiologically tolerable organoiodine compound.

Especially preferably the radio-opaque compositions of the present invention provide an essentially chemically homogeneous distribution of all components within the final radio-opaque composition.

Alternatively, the derivative of a physiologically tolerable organoiodine compound can be used to coat the polymer (e.g. polymer beads or articles comprising the polymer) in order to render the polymer, i.e. articles or compositions comprising it, radio-opaque. This may be achieved, for example, by spraying or dip-coating a polymer-containing component with an organoiodine compound derivative according to the invention in liquid form.

By enzymatically-cleavable derivative of a physiologically tolerable organoiodine compound is meant any derivative which may be cleaved by enzymes particularly enzymes endogenous to a human or animal, e.g. mammalian host, to release physiologically tolerable degradation products. One example is a physiologically tolerable organoiodine compound attached to a physiologically tolerable polymerizable or polymer-philic group (e.g. an acyl group) via an enzymatically cleavable bond such as an ester bond. It is a preferred aspect of the invention that the derivative is an ester of an organoiodine compound. Preferred derivatives include iohexyl hexa-acetate (IHA), Iopamidol penta-acetate, methyl diatrizoate and dimethyl dipamidate. IHA is especially preferred.

The derivatives of organoiodine compounds used in the invention function as contrast media and are freely soluble in non-acrylic monomers and/or polymers. The resulting composition therefore has a chemically homogenous distribution of the organoiodine derivative within the polymer. Such a homogenous composition is advantageous for X-ray monitoring as even very small devices will contain sufficient iodine compound to be detectable. Moreover, homogeneity will also improve the mechanical strength of the composition.

Ideally, the radio-opaque compositions of the invention may comprise 0.5 to 80% by weight, preferably 1 to 50% by weight, e.g. 2 to 20% by weight, particularly 5 to 15% by weight, i.e. around 10% by weight, cleavable derivative of a physiologically tolerable organoiodine compound.

The derivatives can be considered to be prodrugs of the corresponding organoiodine compounds in the sense that cleavage (for example by the body's esterases) releases physiologically tolerable organoiodine compounds.

Preferably the physiologically tolerable organoiodine compound of the invention is an iodinated contrast agent with regulatory approval, which includes diatriozinic acid, iobenguane, iobenzamic acid, iobitriol, iocarmic acid, iocetamic acid, iodamide, iodipamide, iodixanol, iodized oil, iodoalphionic acid, p-iodianiline, o-iodobenzoic acid, iodochlorohydroxyquin, o-iodohippurate sodium, o-iodophenol, p-iodophenol, iodophthalein sodium, iodopsin, iodpyracet, iodopyrrole, iodoquinol, iofetamine ¹²³I, ioglycamic acid, iohexyl, iomeglamic acid, iomeprol, iopamidol, iopanoic acid, iopentol, iophendylate, iophenoxic acid, iopromide, iopronic acid, iopydol, iopydone, iothalamic acid, iotrolan, ioversol, ioxiglimic acid, ioxalic acid, ioxilan and ipodate.

Examples of derivatives for use in the invention are those corresponding to existing water soluble non-ionic contrast agents (for example those listed above) but with the water-solubilising hydroxy groups derivatised such that retention of the organoiodine compound within the polymer is facilitated by increasing its solubility in the polymer and thus the homogeneity of its distribution is also increased and any metabolites produced will correspond to medically approved contrast agents.

The use of such derivatives is especially advantageous as any organoiodine compound released from the polymer, e.g. due to esterase activity of biological fluids, will be in the form of a physiologically tolerable compound or a compound with bio-distribution, bio-elimination and bio-tolerability closely similar to the known and approved contrast agents. Before such exposure to esterase activity, derivatisation with lipophilic groups will moreover serve to reduce any leaching of the organoiodine compound from the polymer. Especially preferred derivatives of physiologically tolerable organoiodine compounds according to the invention include analogues of known non-ionic, monomeric or dimeric organoiodine X-ray contrast agents in which solubilising hydroxyl groups are acylated (e.g. acetylated) or formed into 2,4-dioxacyclopentan-1-yl groups and/or, where the compound is to be polymerizable, in which a carbonyl- or nitrogen-attached ring substituent is replaced by a (meth)acrylamide group or a (meth) acrylamidoalkylamino carbonyl group), or even more preferably the hydroxyl groups are derivatized with biodegradable monomers (e.g. esterified with glycolic acid, lactic acid or E-hydroxycaproic acid).

Examples of conventional non-ionic X-ray contrast agents (i.e. physiologically tolerable organoiodine compounds) which may be modified in this way include: iohexyl, iopentol, iodixanol, iobitridol, iomeprol, iopamidol, iopromide, iotrolan, ioversol and ioxilan. The use of the analogues of the contrast agents with regulatory approval (e.g. in the US, Japan, Germany, Britain, France, Sweden or Italy) is preferred. The use of the analogues of the monomeric contrast agents is particularly preferred. Such analogues may be prepared by esterification of the contrast agent (e.g. by acylation of hydroxyl groups, e.g. acetylation and/or by preparing alkyl esters such as ethyl esters of carboxylic groups). Typical examples of derivatives of physiologically tolerable organoiodine compounds according to the invention (non-polymerizable biodegradable X-ray prodrugs) are shown below:

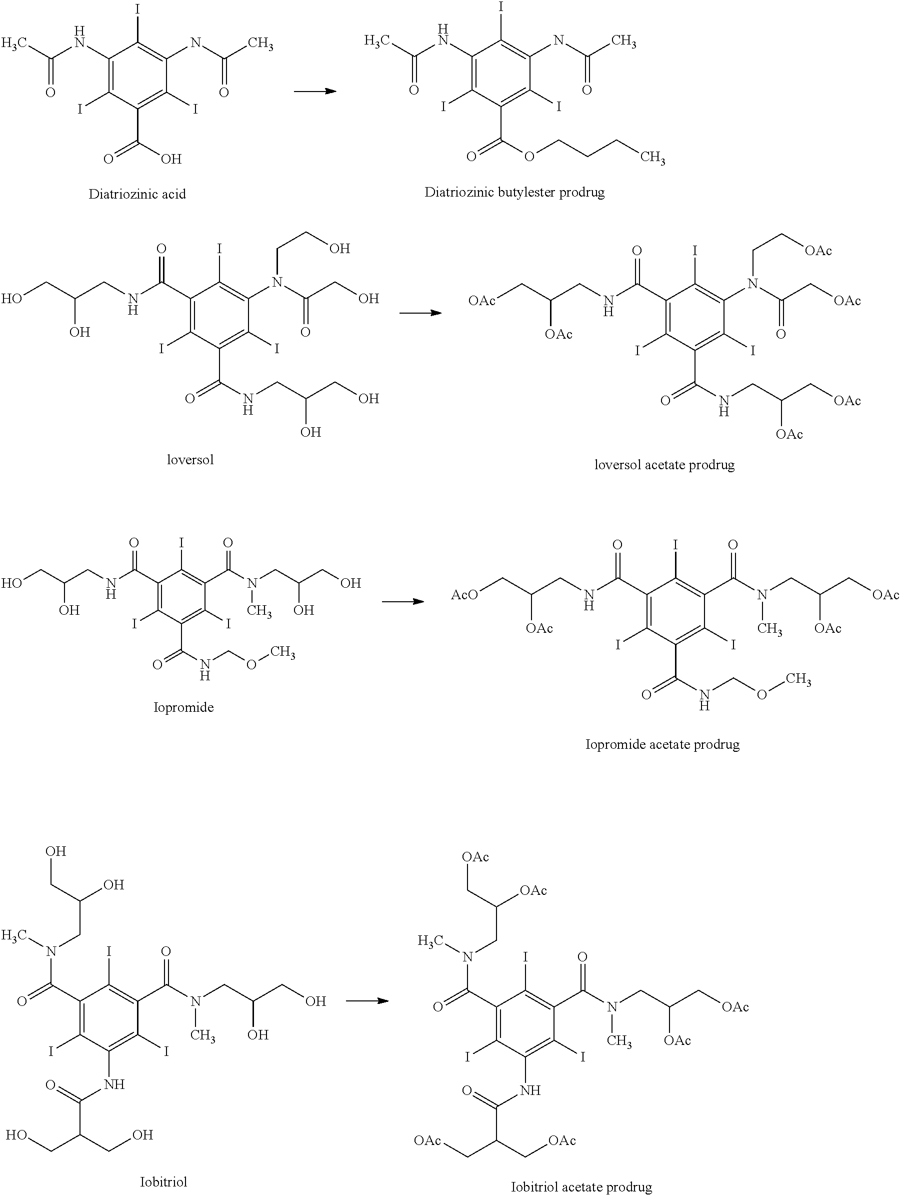

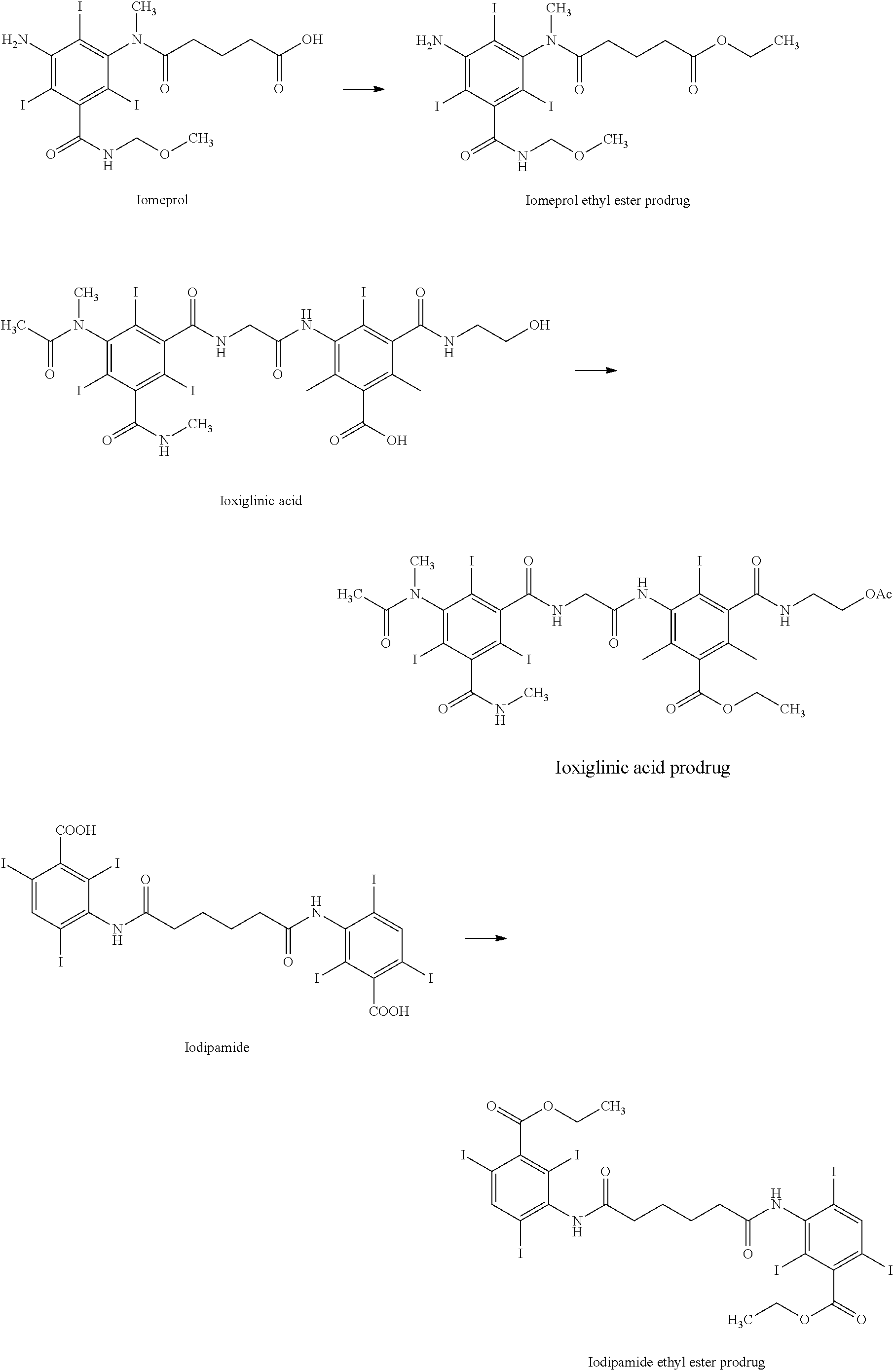

These non-ionic contrast agents can also be derivatized to polymerizable monomer derivatives, by subsequent reaction of an optionally activated alkeneoic acid (e.g. an alkeneoic acid chloride (for example methacrylic acid chloride)), or more preferably derivatized with biodegradable/bioresorbable polymerizable monomers (e.g. esterification with glycolic acid, lactic acid or E-hydroxycaproic acid).

Examples of polymerizable organoiodine compounds include:

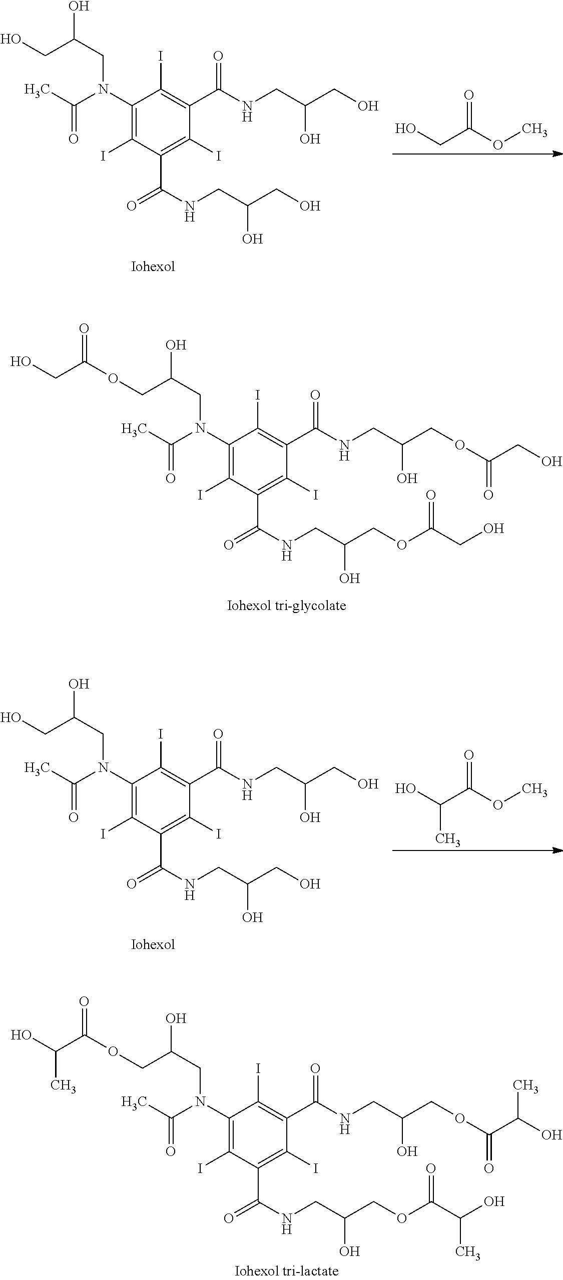

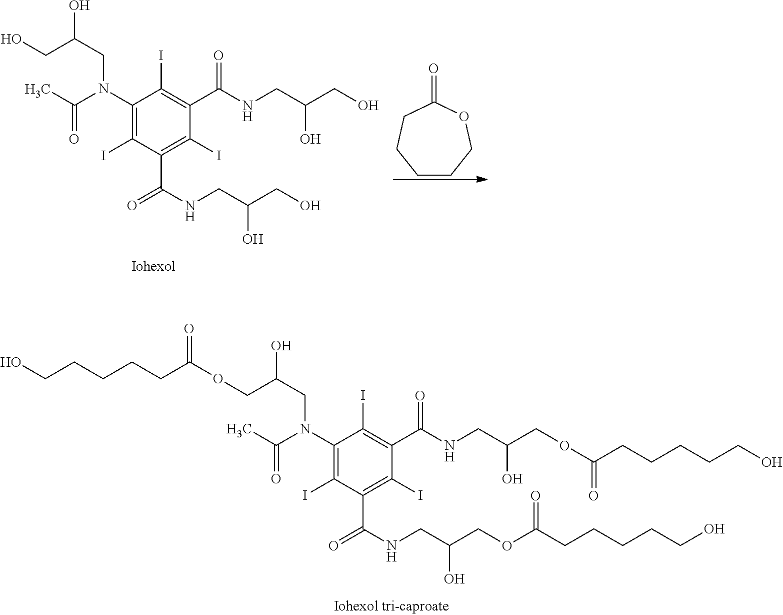

If desired, some or all of the organoiodine compounds may take the form of a cross-linking agent carrying at least two and optionally up to 10 or more polymerizable groups (e.g. esters of glycolic acid, lactic acid, ε-hydroxylhexanoic acid and the like). Generally however such cross-linking agents will constitute only a minor proportion, e.g. up to 20% (on a molar iodine basis) of the total organoiodine compound used, more preferably up to 10%, especially up to 5%. Such cross-linking agents may conveniently be prepared by reacting conventional X-ray contrast agents of the types mentioned above or their aminobenzene precursors (or partly acylated versions of either thereof) with an optionally activated alkeneoic acid (e.g. methacrylic acid chloride) or more preferably an hydroxyalkane carboxylic acid thereof.

Less preferably, the organoiodine compound may be an iodobenzene free from non-polymerizable lipophilic substituents (other than iodine of course), e.g. a simple iodobenzene (such as 1,4-diiodobenzene) or a simple iodoaminobenzene conjugate with (meth)acrylic acid (e.g. methacrylamido-2,4,6-triiodobenzene) or glycolic acid (e.g. glycolamido-2,4,6-triiodobenzene).

Alternatively, the derivative of a physiologically tolerable organoiodine compound according to the invention may be a compound of formula (I):

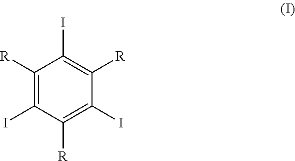

wherein each R group which may be the same or different, comprises an acyloxyalkylcarbonylamino, N-(acyloxyalkyl carbonyl) acyloxyalkylamino, N-acyloxyalkylcarbonyl-N-alkyl-amino, acyloxyalkylaminocarbonyl, bis(acyloxyalkyl)aminocarbonyl, N-acyloxyalkyl-N-alkyl-aminocarbonyl, alkoxyalkylaminocarbonyl, N-alkyl-alkoxyalkylaminocarbonyl, bis(alkoxyalkyl)amino-carbonyl, alkoxyalkylcarbonylamino, N-alkyl-alkoxyalkylcarbonylamino or N-alkoxyalkylcarbonyl-alkoxyalkylamino group or a triiodophenyl group attached via a 1 to 10 atom bridge (preferably composed of bridging atoms selected from O, N and C) optionally substituted by an acyloxyalkyl, acyloxyalkylcarbonyl, acyloxyalkylamino, acyloxyalkylcarbonylamino, acyloxyalkylaminocarbonyl, alkoxyalkyl, alkoxyalkylcarbonyl, alkoxyalkylamino, alkoxyalkylcarbonylamino, or alkoxyalkylaminocarbonyl group or by a polymerizable group, e.g. a hydroxyalkane, (meth)acrylate or (meth)acrylamide group, or one or two R groups is/are a polymerizable group, e.g. a hydroxyalkane, (meth)acrylate or (meth)acrylamide group, optionally attached via a 1 to 10 atom bridge, e.g. an alkylaminocarbonyl or alkylcarbonylamino bridge; or where one R group is a polymerizable group, one or both of the remaining R groups may be an alkylamino, bisalkylamino, alkylcarbonylamino, N-alkyl-alkylcarbonylamino, alkylaminocarbonyl or bis-alkyl-aminocarbonyl group, (e.g. an acetylamino group). In such compounds, any alkyl or alkylene moiety preferably contains 1 to 6 carbon atoms, especially 2 to 4 carbon atoms and any bridge optionally comprises oxygen and/or nitrogen atoms, especially one or two nitrogen atoms. Moreover, two alkoxy groups in such compounds, especially groups attached to neighbouring carbon atoms, may be fused to form a cyclic bis-ether, preferably containing two ring oxygens and three ring carbons, e.g. as a 2,4-dioxa-3,3-dimethyl-cyclopentan-1-yl group. In general, it is preferred that two R groups are carbonyl-attached and that one is nitrogen-attached to the iodobenzene ring.

The non-acrylic polymer of the composition of the invention will be selected according to the intended use of the radio-opaque composition and thus will be apparent to the skilled person. Examples of suitable polymers are; polystyrene, poly(lactic acid) (PLA), poly(ε-caprolactone) (PCL), poly(glycolic acid) (PGA), poly(lactide-co-glycolide) (PLGA), poly(dioxanone), poly(glycolide-co-trimethylene carbonate), poly(vinyl alcohol) (PVA), poly(vinylpyrrolidine), poly(hydroxybutarates), poly(hydroxyvalerate), poly(sebaic acid-co-hexadecandioic acid anhydride), poly(orthoester), poly(caprolactams), poly(acrylamides), poly(terphthalate), polyether block amides (PEBA), poly(urethane) etc. Polymer blends, alloys, homopolymers, random copolymers, block copolymers and graft copolymers are also suitable.

Bio-stable/bio-compatible polymers such as polyamides, polyanhydrides, polycarbonates, polyesters, polyethers, poly(hydrocarbons), polyurethanes, polysulfones and polysiloxanes, and their copolymers are especially preferred, as are bio-absorbable polymers such as polylactide, polyglycolide, polycaprolactone, poly(dioxanone) tyrosine and their copolymers. Polyhydroxyalkanocarboxylic acids as poly(lactide-co-glycolide) polymers are preferred due to their biocompatibility and biodegradability properties.

Preferably the polymer comprises polyesters such as poly(L-lactide), poly(D,L-lactide), poly(caprolactone), poly(glycolic acid), poly(lactide-co-glycolide), poly(lactide-co-caprolactone), poly(glycolide-co-caprolactone), poly(L-lactide-co-caprolactone-co-glycolide), polytrimethylene carbonate, poly(3-hydroxybutyrate), poly(3-hydroxyvalerate), poly(4-hydroxybutyrate), poly(dioxanone) polyamides such as poly(caproamide), poly(hexamethylene adipamide), poly(p-phenyleneterephtalamide), polyhydrocarbones such as poly(ethylene), poly(propylene), poly(1-hexene), poly(1-hexene-co-4-methyl-1,4-hexadiene), poly(tetrafluoroethylene), poly(vinyl alcohol), polyacetals such as poly(formaldehyde), polyketals, polyglycols, polyurethanes, segmented polyurethanes, polyanhydrides, polyphosphazenes, polysulfones, silicones, ABS resins, natural polymers such as collagen, fibrin, polysaccharides such as chitosan.

It is especially preferred that the polymer of the composition is a biodegradable or biocompatible (e.g. physiologically tolerable) polymer.

The radio-opaque compositions of the present invention may additionally comprise a medical agent, particularly for medical applications. Such agents are included in several conventional radio-opaque compositions and may be used in similar concentrations in the compositions of the invention.

The medical agents may be selected from a wide variety of groups, depending on the device for which they are intended and the corresponding organ. Agents (for example for use in stents or stent systems) include anti-proliferative agents (paclitaxel and the like), immunosuppressive agents (dexamethasone, rapamycin, tacromilus, mycophenolic acid and the like), anti-inflammatory agents (aspirin, ibuprofen, naproxen and the like) anti-matrix metalloproteinase, lipid lowering agents (simvastatin, lovastatin, pravastatin and the like), anti-thrombotic agents and antiplatelet agents (e.g. clopidogrel, ticlodipine, dipyridamole, epoprostenole, iloprostenole, argatroban and the like). The biocompatible/bioresorbable polymer may comprise antibiotics or antiseptics, e.g. gentamicin, colistin, erythromycin, clindamicin, penicillins, norfloxacin, chloramphenicol etc.

The medical agents are usually added to medical devices by different coating processes (e.g. air knife, immersion, curtain coating and the like) or matrix loading. Coating processes are used most frequently, but the agents are only deposited onto the device surface and will be released rapidly to the biological surroundings. Release of a medical agent over a prolonged period can be obtained by matrix loading, a process where the agents are incorporated into the medical device. Preferably, in order to maintain the mechanical strength of the device (especially when the medical agent contains hydrophilic groups such as alcohols, carboxylic acid etc.) the medical agents may be in the form of a lipophilic ester, such as an acyl derivative e.g. an acetyl ester, and/or e.g. ethyl esters, or any biodegradable prodrug depending on the chemical nature of the agent, whereby to allow its release from the composition over a prolonged period as a result of esterase activity in the physiological fluids contacting the composition after implantation. A typical example of such derivatives are gentamycin poly-acetate, dipyridamol acetate, epoprostenol ethyl ester and the like.

Some examples of typical prodrugs for use in medical polymers are shown below:

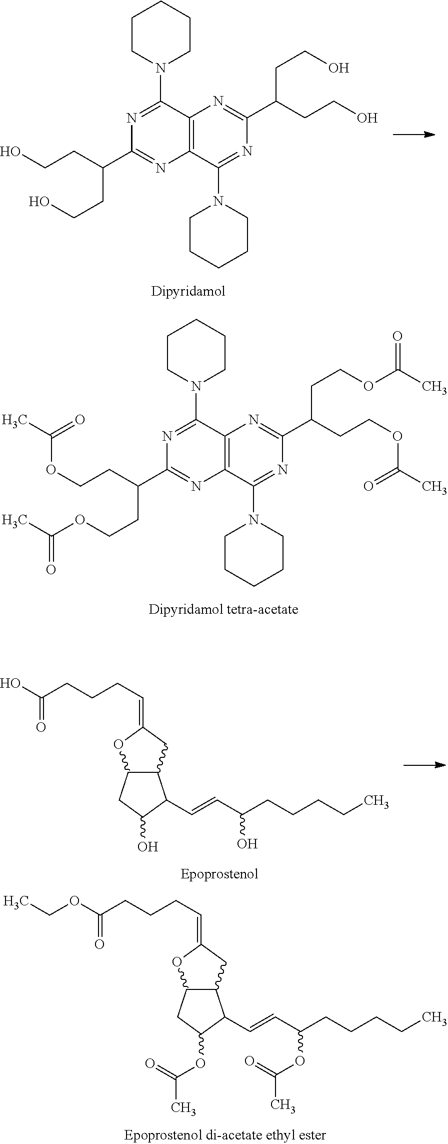

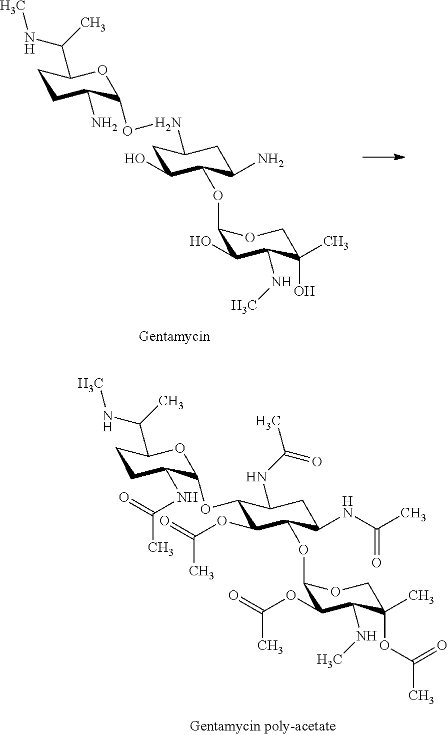

Alternatively, such agents may be copolymerized into the polymer by incorporating a polymerizable hydroxyalkane and/or ethylenically unsaturated bond coupled via an ester group to the drug moiety, whereby again to release the medical agent over a prolonged period as a result of esterase activity. Preferably, the medical agents should be hydrophilic to prevent rapid release and to prevent infection after surgery.

The present invention further provides a process for producing a radio-opaque composition as herein described wherein said process comprises combining a non-acrylic monomer composition with a cleavable, preferably enzymatically-cleavable, derivative of a physiologically tolerable organoiodine compound and carrying out a polymerization reaction. The derivative of a physiologically tolerable organoiodine compound may or may not take part in the polymerization reaction, i.e. it may be co-polymerizable with the non-acrylic monomer, but is not necessarily so.

The non-acrylic monomer composition will comprise at least one non-acrylic polymerizable monomer, generally a monomer containing a hydroxyalkane group and/or ethylenically unsaturated bonds, optionally a polymerization initiator, and optionally a cross-linking agent. The polymerization initiator and cross-linking agent may if desired be added to the monomer mixture during preparation of the radio-opaque composition for use.

Most biodegradable polymers are synthesized by condensation polymerization (ring opening polymerization. An example of this is shown below:

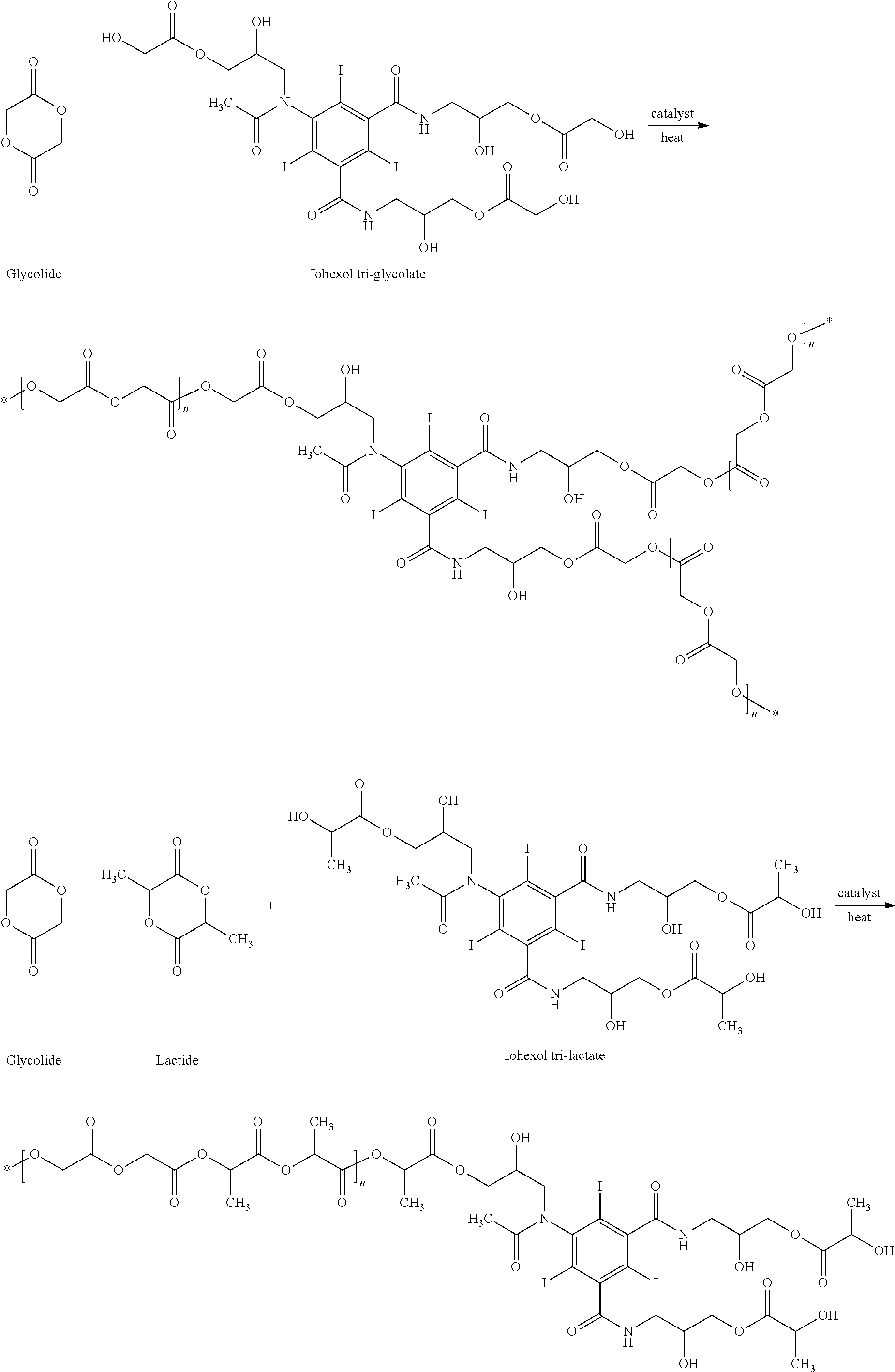

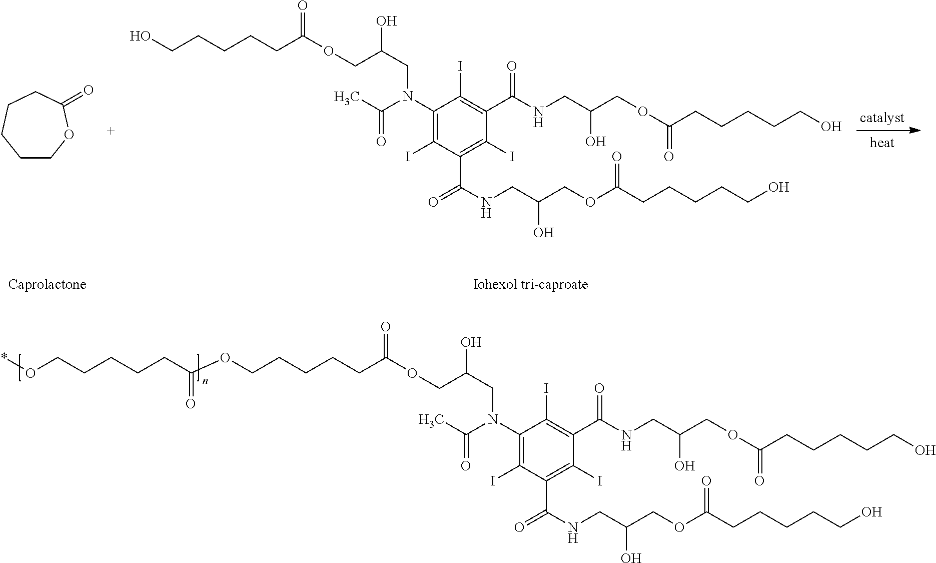

Other biocompatible polymers (polyethylene, e.g. HDPE or polyvinylchloride etc.) are synthesized by addition polymerization, and the non-acrylic organoiodine monomers can also be copolymerized with these polymers to give copolymers of biodegradable contrast agents.

The polymerization initiator, where present, is preferably a physiologically tolerable initiator of polymerization of ethylenically unsaturated monomers, e.g. N,N-dimethyl-p-toluidine, N,N-dimethylaminobenzyl alcohol (DMOH) or N,N-dimethylaminobenzyl oleate (DMAO), or, for ring-opening polymerizaton typical initiators are tin-2-ethylhexanoate (SnOct), dibutyltin dilaurate, bismuth(III)-n-hexanoate, bismuth subsalicylate, stannaous octoate, hexamethyl-cyclotrisiloxane and the like. The initiator typically constitutes 0.01 to 10% wt. of the monomer composition, preferably 0.1 to 5% wt., more preferably 0.5 to 2% wt., especially 0.1 to 1% wt.

If a cross-linking agent (e.g. an organoiodine compound containing two or more polymerizable groups, polyethyleneglycols etc.) is present in the monomer composition, this preferably constitutes up to 5% wt. of the composition, more preferably, 2% wt., especially 0.1 to 1% wt. of the composition.

The polymerization temperature can vary over a large range. Preferably the polymerization temperature is in the range of 50-250° C.; more preferably 100-175° C.; especially preferably 125-175° C.

The polymerization reaction time can also vary over a large range. Preferably the reaction time is in the range from 4 hours to 5 days, more preferably 6 hours to 2 days.

All these parameters affect the physical and chemical properties of the polymer formed i.e. molecular weight, monomer content etc.

In a preferred process, the cleavable non-polymerizable derivatives of organoiodine contrast agents are added (preferably with mixing) to the biodegradable/biocompatible polymers (which are, for example, in solution, bead or powder form) and they are typically heated to a melt under stirring. The polymer blend can be extruded and/or moulded directly, or alternatively cooled to leave the polymer composition as a solid (e.g. a powder or a film) with the contrast agent homogenously distributed therein.

The non-acrylic polymer and organoiodine compound may be dissolved in a suitable solvent, e.g. dichloromethane, chloroform, dimethylsulfoxide, dimethylformamide, toluene and the like. The blend can be evaporated under reduced pressure to leave the polymer composition as a homogenous solid.

More preferably the polymer blend can be spray-dried to leave polymer beads with organoiodine compounds homogenously distributed therein.

The polymer blend/compositions may be processed similar to any engineering thermoplastic in that they can be melted down and formed into fibres, rods and moulded parts. Final parts can be extruded, injection moulded, compression moulded, or solvent spun or cast. In some circumstances the primary processing may be followed by subsequent machining into final parts.

The radio-opaque compositions of the present invention have a variety of uses. In particular they will be used in the production of radio-opaque articles, for example for coating articles or moulding articles therefrom. The use of the composition of the invention in the production of radio-opaque articles and the articles themselves form a further aspect of the present invention.

The article may be a medical device. For example medical stents, implantable devices for orthopaedics, tissue engineering, dental applications, gastric lap bands, drug delivery, cancer treatment, other cardiovascular applications, non-cardiovascular stents such as biliary, oesophagus, vaginal, lung-trachea/bronchus, and the like. In addition, the contrast media are suitable for use in producing implantable, radio-opaque discs, plugs, and other devices used to track regions of tissue removal, for example, in the removal of cancerous tissue and organ removal, as well as staples and clips suitable for use in wound closure, attaching tissue to bone and/or cartilage, stopping bleeding, tubal ligation, surgical adhesion prevention and the like.

Furthermore, in some preferred embodiments of the invention, the present contrast media may be advantageously used in making various orthopaedic devices including, for example radio-opaque biodegradable screws, radio-opaque biodegradable suture anchors, and the like for use in applications including the correction, prevention, reconstruction, and repair of the anterior cruciate ligament (ACL), the rotator cuff/rotator cup, and other skeletal deformities.

Other devices, which advantageously can be made radio-opaque with the present invention includes devices for use in tissue engineering. Examples of suitable devices include tissue engineering scaffolds and grafts (such as vascular grafts, grafts or implants used in nerve regeneration). The present contrast agents may also be added to polymers used to form a variety of devices effective for use in closing internal wounds. For example biodegradable sutures, clips, staples, barbed of mesh sutures, implantable organ supports, and the like, for use in various surgery, cosmetic applications, and cardiac wound closures can be formed.

Various devices finding use in dental applications can advantageously be made using radio-opaque compositions according to preferred aspects of the present invention. For example devices for guided tissue regeneration, alveolar ridge replacement for denture wearers, and devices for the surgeon/dentist can ascertain the placement and continuous function of such implants by simple X-ray imaging.

The present contrast agents are also useful in the production of gastric lap bands for use in the treatment of obesity. The production of radio-opaque lap bands allows for more effective monitoring of the devices in the human body, and more effective treatment of obesity.

In addition to intravascular stents and non-cardiovascular stents, the present polymers are useful in a number of other cardiovascular and vascular devices. For example valves, chordae tendinea replacements, annuloplasty rings, leaflet repair patches, vascular grafts, vascular tubes, patches for septal defects, arterial and venous access closure devices (plugs), and the like can be used in replacement repair of heart valves, tubes and the like.

More specifically these include medical/surgical tubing e.g. for renal and celial arteriography or for producing mini-balloon catheters, protective sheeting, surgeons' gloves, intubation sets, heart catheters, stomach tubes, nasal tubes, thoracic catheters, string, mesh, suture, braid, stent, catheter, cannula, plug, constrictor, bone anchor, plate, rod, seed, capsule sheet, tubes, guide wires, shunts, screws, pins, prostheses, films, sponges, balloons, needles, markers, stylets, membranes, autotransfusion devices, blood filters, blood gas exchange devices, blood pumps, blood temperature monitors, bone growth stimulators, breathing circuit connectors, bulldog clamps, cannulae, grafts, implantable pumps, impotence and incontinence implants, intra-ocular lenses, leads, lead adapters, lead connectors, nasal buttons, orbital implants, cardiac insulation pads, cardiac jackets, clips, covers, dilators, dialysers, disposable temperature probes, domes, drainage products, drapes, ear wicks, electrodes, embolic devices, oesophageal stethoscopes, fracture fixation devices, gloves, guide wires, hemofiltration devices, hubs, intra-arterial blood gas sensors, intracardiac suction devices, intrauterine pressure devices, nasal septal splints, nasal tampons, needles, ophthalmic devices, oxygenators (both sheet and tubular forms of membrane oxygenators), PAP brushes, periodontal fibre adhesives, pessary, pins, retention cuffs, screws, sheeting, sponges, staples, stomach ports, surgical instruments, transducer protectors, urethral stents, vaginal contraceptives, valves, vessel loops, water and saline bubbles, achtabular cups, annuloplasty ring, aortic/coronary locators, artificial pancreas, balloons, batteries, bone cement, breast implants, cardiac materials, such as fabrics, felts, films, markers, mesh, patches, cement spacers, cochlear implant, defibrillators, generators, orthopaedic implants, pacemakers, patellar buttons, penile implant, pledgets, plugs, plates, ports, prosthetic heart valves, sheeting, shunts, stylets, umbilical tape, valved conduits, surgical-use cotton and vascular access devices.

Preferably the medical device according to the invention is selected from catheters, tubes, strings, meshes, sutures, cotton, stents, cannulae, plugs, plates, rods, guide wires, shunts, screws, pins, prostheses, balloons, needles, clips, and staples.

Other preferred devices are scaffolds, drug delivery systems, endoprostheses of heart valves, endoprostheses of ligaments, tendons and muscles and dental filling composites.

In a further embodiment the present invention provides articles comprising a radio-opaque composition wherein said radio-opaque composition comprises a cleavable, preferably enzymatically-cleavable derivative of a physiologically tolerable organoiodine compound and a polymer, preferably a non-acrylic and/or biodegradable polymer. The articles according to this embodiment can be any for which X-ray monitoring can be envisaged. Preferred articles according to this embodiment of the invention include toys or toy components (e.g. building blocks, eyes and buttons for dolls and cuddly animals) and other things which children are likely to ingest. Thus, a further aspect of the invention is a toy comprising a radio-opaque composition wherein said radio-opaque composition comprises a polymer and a cleavable, preferably enzymatically-cleavable, derivative of an organoiodine compound.

Moreover, radio-opaque compositions comprising a polymer and the polymer-soluble organoiodine compounds described herein are also potentially useful in situations when the attenuation of x-ray radiation is desired, e.g. in panels in radiography departments or protective shields etc. Radiation-protective equipment comprising an organoiodine compound dissolved in a polymer provides a further aspect of the invention.

The invention will now be described further with reference to the following non-limiting Examples. Parts and percentages are by weight unless otherwise indicated.

### EXAMPLE 1

### Stability of Iohexyl Hexa-Acetate in Human Plasma

A stock solution of iohexyl hexa-acetate was prepared by adding 100 mg IHA to a 100 ml volumetric flask, followed by 1.0 ml DMSO and deionised water to 100 ml, giving a final concentration of 1.0 mg/ml. The plasma solution was prepared by adding 1.61 ml of the IHA stock solution to 3.39 ml of citrated bovine plasma, giving a final concentration of 300 μM. The citrated human plasma was incubated at 37° C., and 0.25 ml plasma removed at 1, 2, 3, 4, 6, 8, 24, 30 and 48 h. Proteins were discarded from the samples by centrifugal filtration and the resulting filtrates analyzed by HPLC. The concentrations of iohexyl hexa-acetate and iohexyl from 0 to 48 h are plotted in FIG. 1.

As shown in FIG. 1, the concentration of iohexyl is constant the first 8 h, then the concentration increases dramatically from 8 to 48 h. At the same time the change in iohexyl hexa-acetate concentration follows and opposite trend. The iohexyl hexa-acetate concentration starts to decrease immediately after incubation at 37° C., and after 24 h it is no longer possible to detect any IHA left in the plasma.

### EXAMPLE 2

### Preparation of poly(L-lactide-co-caprolactone-co-glycolide, 70:20:10) with iohexyl hexa-acetate homogenously distributed therein

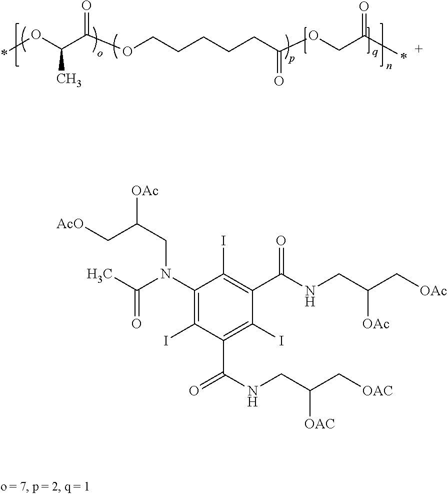

Iohexyl hexa-acetate (10 mg) was added to a stirred solution of poly(L-lactide-co-caprolactone-co-glycolide, 70:20:10)(90 mg) in CH₂Cl₂ (2.0 ml) and heated at 40° C. for 30 minutes. The mixture was cooled to room temperature, evaporated in vacuo to leave the product as a white crystalline solid.

### EXAMPLE 3

### Preparation of poly(ε-caprolactone) with iohexyl hexa-acetate homogenously distributed therein

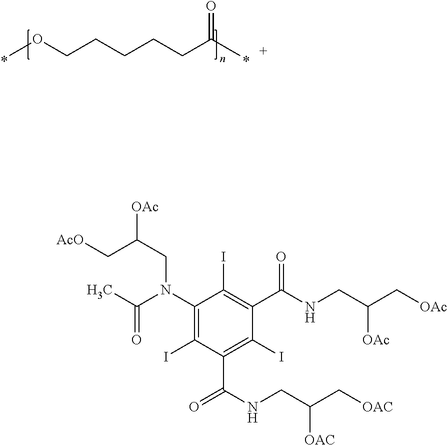

Iohexyl hexa-acetate (0.10 g) was added to a stirred solution of poly(ε-caprolactone) (0.90 g) in CH₂Cl₂ (2.0 ml) and heated at 40° C. for 30 minutes. The mixture was cooled to room temperature, evaporated in vacuo to leave the product as a white crystalline solid.

### EXAMPLE 4

### Preparation of poly(lactide-co-glycolide, 50:50) with iohexyl hexa-acetate homogenously distributed therein

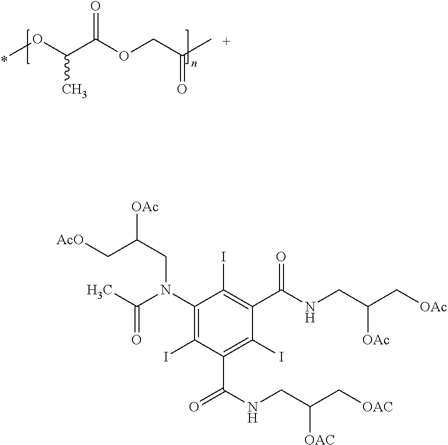

Iohexyl hexa-acetate (10 mg) was added to a stirred solution of poly(lactide-co-glycolide, 50:50) (90 mg) in CH₂Cl₂ (2.0 ml) and heated at 40° C. for 30 minutes. The mixture was cooled to room temperature, evaporated in vacuo to leave the product as a white crystalline solid.

### EXAMPLE 5

### Preparation of Poly(DL-lactide) with iohexyl hexa-acetate homogenously distributed therein

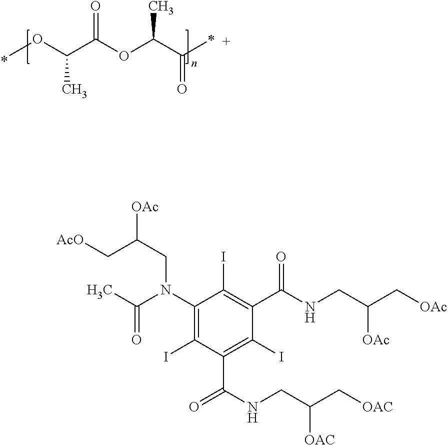

Iohexyl hexa-acetate (0.10 g) was added to a stirred solution of poly(DL-lactide) (0.90 g) in CH₂Cl₂ (5.0 ml) and heated at 40° C. for 30 minutes. The mixture was cooled to room temperature and the product precipitated with MeOH (5.0 ml). The precipitate was filtered off and dried in vacuo to leave the product as a white crystalline solid.

### EXAMPLE 6

### Synthesis of Iopamidol Pentaacetate

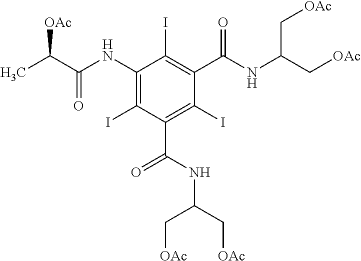

Acetic anhydride (31.2 g, 0.30 mol) was added dropwise to a suspension of iopamidol (20.0 g, 25.7 mmol) in pyridine (100 ml) at room temperature. The reaction mixture was stirred for 48 h, then poured into water (isotonic, 0.8 L) and the compound precipitated out of the solution. The precipitate was filtered off, and the residue recrystallized to leave the title compound as a white crystalline solid (22.1 g, 86%). ¹H-NMR (DMSO-_{d6}) δ 10.13 (s, 1H), 8.92 (t, 1H), 8.81 (d, 1H), 5.26-5.20 (m, 1H), 4.36-4.30 (m, 2H), 4.17-4.12 (m, 8H), 3.31 (s, 2H), 2.10 (d, 2H), 2.03 (br s, 12H), 1.51 (d, 2 H).

### EXAMPLE 7

### Synthesis of Methyl Diatrizoate

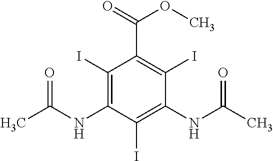

Methyliodide (1.55 g, 10.98 mmol) was added to a mixture of diatrizoic acid (5.0 g, 8.14 mmol) and Cs₂CO₃ (2.65 g 8.14 mmol) in DMSO (15 ml) and stirred at room temperature for 4 h. Water (70 ml) was added to the reaction mixture and a white solid precipitated out, filtered off and the residue separated with flash chromatography (SiO₂, CH₂Cl₂) to leave the title compound as a white solid (3.25 g 62.5%). ¹H-NMR (DMSO-_{d6}) δ10.04 (s, 2H), 3.31 (s, 3H), 2.01 (s, 6H)

### EXAMPLE 8

### Synthesis of Dimethyl Iodipamidate

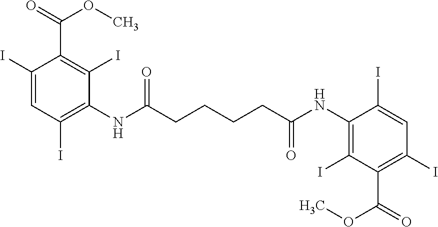

Methyliodide (0.074 g, 0.52 mmol) was added to a mixture of iodipamide (0.20 g, 0.17 mmol) and Cs₂CO₃ (0.23 g, 0.70 mmol) in DMF (3 ml) and stirred at room temperature for 24 h. The reaction mixture was evaporated in vacuo and the residue separated with flash chromatography (SiO₂, CH₂Cl₂) to leave the title compound as a white solid (0.15 g 75%). ¹H-NMR (CDCl₃) δ 8.49 (s, 2H), 3.96 (s, 6H), 1.89 (br s, 4H). 1.40 (br s, 4H)

### EXAMPLE 9

### Compression Moulding of PLLA (poly-L-lactide) Test Bars Containing Iohexyl Hexaacetate

Iohexyl hexaacetate was mixed with PLLA beads to give powder mixtures with 2, 5, 10, 15 and 20 wt % contrast agent. The mixtures were then compression moulded at 200° C. for 2 minutes, allowed to cool to room temperature and cut into PLLA specimens with dimension 50 mm×5 mm×2 mm. The specimens were annealed at 70° C. to leave the final specimens. The samples were cut into pieces of 150 mg, dissolved in CH₂Cl₂ (5.0 ml) and analyzed by HPLC. The chromatograms showed no degradation of iohexyl hexaacetate.

### EXAMPLE 10

### Determination of Mass Loss of PLLA Specimens Containing Iohexyl Hexaacetate

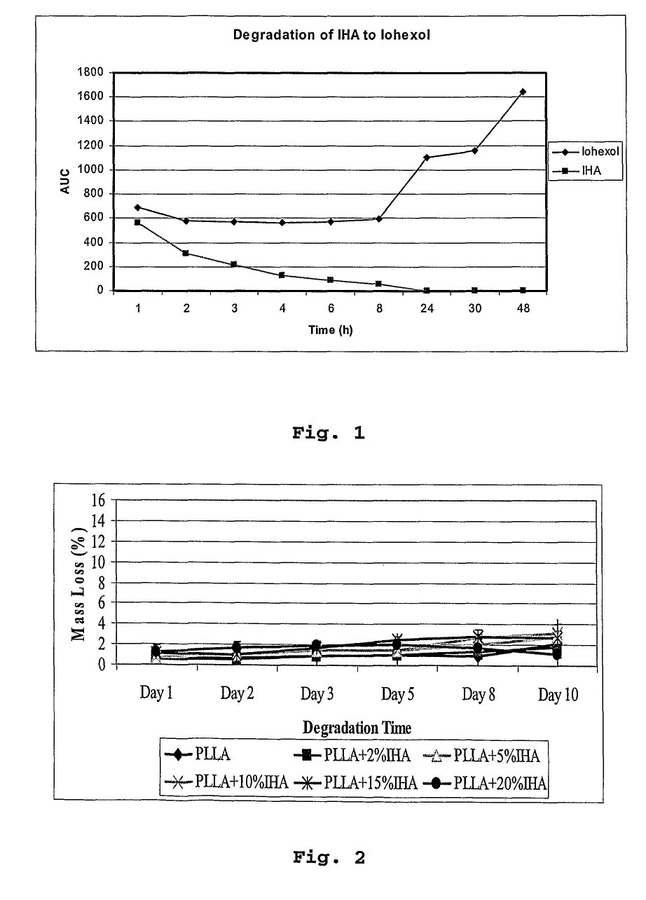

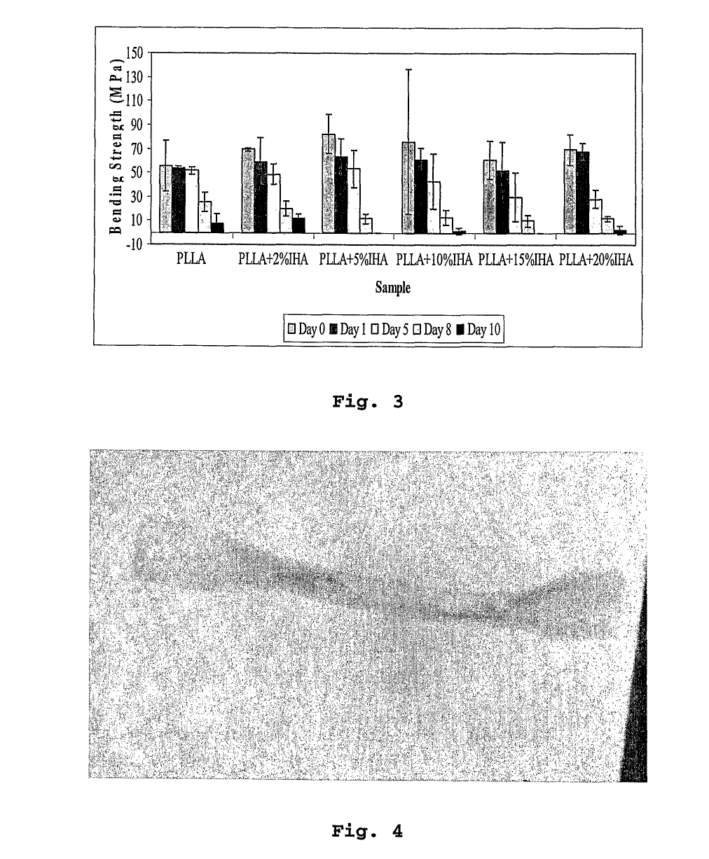

The mass loss of PLLA specimens containing 2, 5, 10, 15 and 20 wt % of iohexyl hexaacetate were determined. Three PLLA specimens for each concentration were incubated in PBS (phosphate buffered saline) at 37° C., and the mass loss was determined by weighing at day 1, 2, 3, 5, 8 and 10. The result is outlined in FIG. 2. There was no significant difference in mass loss between pure PLLA and PLLA with iohexyl hexaacetate added.

### EXAMPLE 11

### Mechanical Testing of PLLA Specimens Containing Iohexyl Hexaacetate

The bending strength of PLLA specimens containing 2, 5, 10, 15 and 20 wt % of iohexyl hexaacetate were determined. Three PLLA specimens for each concentration were incubated in PBS at 37° C., and bending strength at day 1, 5, 8 and 10 determined by four point bending testing. The result is outlined in FIG. 3. There was no significant difference in bending strength between pure PLLA and PLLA with added iohexyl hexaacetate.

### EXAMPLE 12

### Dip Coating of PLLA Specimen

A pure PLLA specimen was dipped in a saturated solution of iohexyl hexaacetate in CH₂Cl₂. The specimen was dried in vacuum at room temperature for 2 h, before the dip coating process was repeated. The PLLA coated specimen was dried in vacuum at room temperature overnight. The PLLA specimen was visualized by X-ray, seen in FIG. 4.

### EXAMPLE 13

### Solvent Casting of Polycaprolactone Containing Iohexyl Hexaacetate

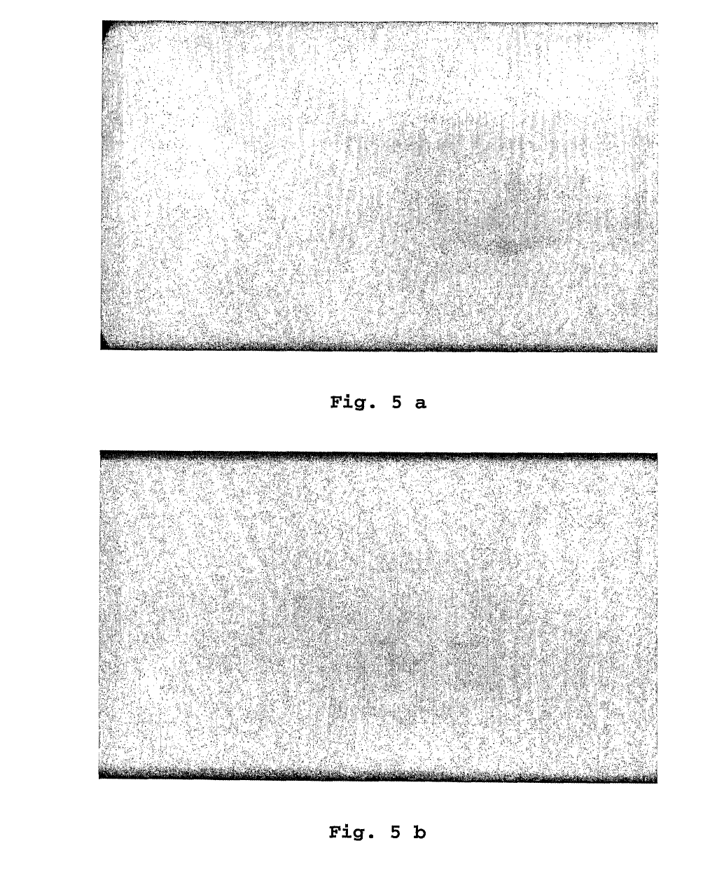

A solution of polycaprolactone (9.0 g) and iohexyl hexaacetate (1.0 g) in CH₂Cl₂ (10 ml) was stirred at 60° C. for 10 minutes, then 0.5, 1.0 and 1.5 ml of the polymer solution was transferred to three vials and dried at 40° C. for 2 h. The polycaprolactone films containing iohexyl hexaacetate were visualized with X-ray, seen in FIG. 5 a (0.5 ml), 5 b (1.0 ml) and 5 c (1.5 ml).

### EXAMPLE 14

### Injection Moulding of Polypropylene Specimens Containing Iohexyl Hexaacetate

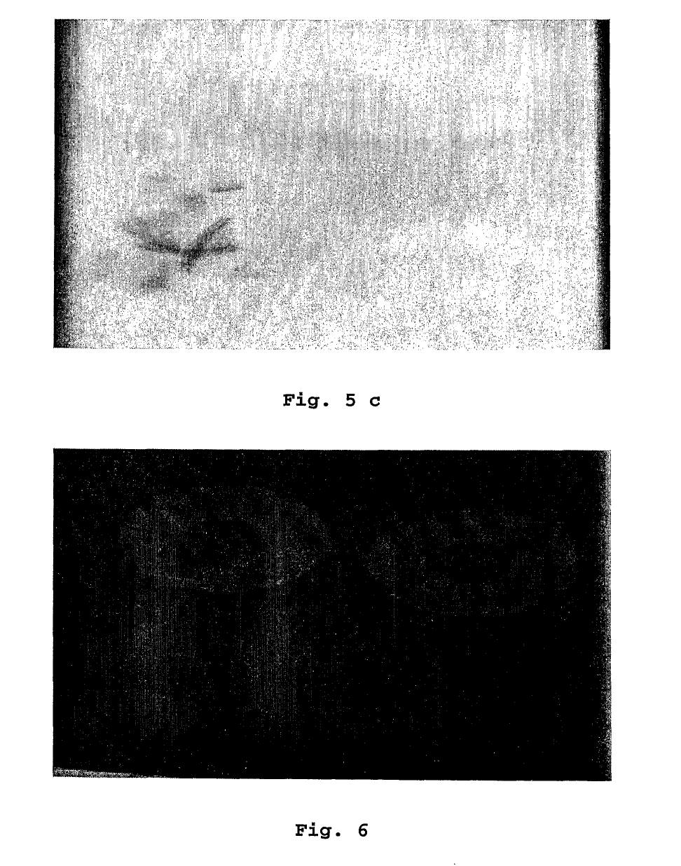

Iohexyl hexaacetate (10 g) was mixed with polypropylene beads (SABIC® RA 12 MN 40) (90 g) and the powder mix injection moulded (DEMAG ERGOTech 25-80, screw temperature 210° C.) to leave polypropylene specimens containing 10 wt % iohexyl hexaacetate. The injection moulded polypropylene specimens with 10 wt % iohexyl hexaacetate were visualized with by X-ray, seen in FIG. 6.

### EXAMPLE 15

### Injection Moulding of Polyamide Specimens Containing Iohexyl Hexaacetate

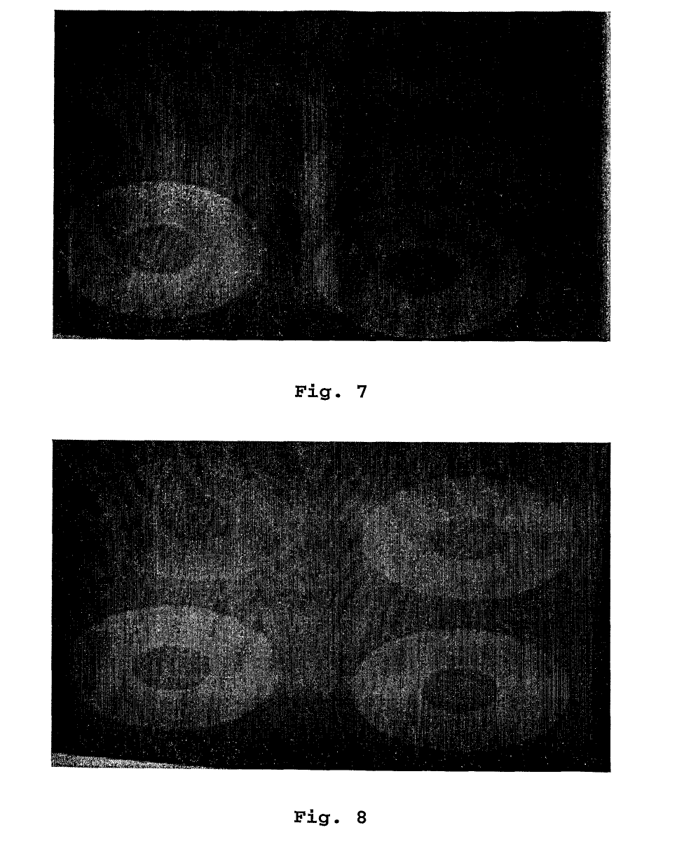

Iohexyl hexaacetate (10 g) was mixed with polyamide beads (PA6) (90 g) and the powder mix injection moulded (DEMAG ERGOTech 25-80, screw temperature 240° C.) to leave polyamide specimens containing 10 wt % iohexyl hexaacetate. The injection moulded polyamide specimens with 10 wt % iohexyl hexaacetate were visualized with X-ray, seen in FIG. 7.

### EXAMPLE 16

### Injection Moulding of HDPE (High Density Poly-Ethylene) Specimens Containing Iohexyl Hexaacetate (IHA)

Iohexyl hexaacetate (10 g) was mixed with HDPE (HMA016 ExxonMobil) (90 g) and the powder mix injection moulded (DEMAG ERGOTech 25-80, screw temperature 180° C.)) to leave HDPE specimens containing 10 wt % iohexyl hexaacetate. The injection moulded HDPE specimens with 10 wt % iohexyl hexaacetate were visualized with X-ray, seen in FIG. 8.

### EXAMPLE 17

### Emulsion Polymerization of PS Beads Containing IHA

A solution of aqueous 1% PVP K90 (500 ml) in a four-necked round-bottom flask is heated to 70° C. with mechanical stirring. A solution of vinylbenzene (70.0 g, 672.1 mmol), iohexyl hexaacetate (30.0 g, 27.9 mmol) and benzoyl peroxide (3.25 g, 13.4 mmol) is added dropwise and the emulsion stirred at 70° C. for 24 h, cooled to room temperature and the PS beads filtered off and the solid lyophilized to leave PS beads with iohexyl hexaacetate incorporated.

1. A radio-opaque composition comprising a cleavable, preferably enzymatically-cleavable, derivative of a physiologically tolerable organoiodine compound and a non-acrylic polymer wherein said derivative is dissolved in said non-acrylic polymer.

2. The composition as claimed in claim 1 wherein said derivative is a lipophilic ester of said organoiodine compound.

3. The composition as claimed in claim 1 wherein said organoiodine compound is selected from diatriozinic acid, iobenguane, iobenzamic acid, iobitriol, iocarmic acid, iocetamic acid, iodamide, iodipamide, iodixanol, iodized oil, iodoalphionic acid, p-iodianiline, o-iodobenzoic acid, iodochlorhydroxyquin, o-iodohippurate sodium, o-iodophenol, p-iodophenol, iodophthalein sodium, iodopsin, iodpyracet, iodopyrrole, iodoquinol, iofetamine ¹²³I, ioglycamic acid, iohexyl, iomeglamic acid, iomeprol, iopamidol, iopanoic acid, iopentol, iophendylate, iophenoxic acid, iopromide, iopronic acid, iopydol, iopydone, iothalamic acid, iotrolan, ioversol, ioxiglimic acid, ioxalic acid, ioxilan and ipodate.

4. The composition as claimed in claim 1 wherein said derivative of a physiologically tolerable organoiodine compound is a compound of formula (I): 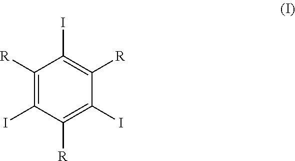
wherein each R group which may be the same or different, comprises an acyloxyalkylcarbonylamino, N-(acyloxyalkyl carbonyl)acyloxyalkylamino, N-acyloxyalkylcarbonyl-N-alkyl-amino, acyloxyalkylaminocarbonyl, bis(acyloxyalkyl)aminocarbonyl, N-acyloxyalkyl-N-alkyl-aminocarbonyl, alkoxyalkylaminocarbonyl, N-alkyl-alkoxyalkylaminocarbonyl, bis(alkoxyalkyl)amino-carbonyl, alkoxyalkylcarbonylamino, N-alkyl-alkoxyalkylcarbonylamino or N-alkoxyalkylcarbonyl-alkoxyalkylamino group or a triiodophenyl group attached via a 1 to 10 atom bridge (preferably composed of bridging atoms selected from O, N and C) optionally substituted by an acyloxyalkyl, acyloxyalkylcarbonyl, acyloxyalkylamino, acyloxyalkylcarbonylamino, acyloxyalkylaminocarbonyl, alkoxyalkyl, alkoxyalkylcarbonyl, alkoxyalkylamino, alkoxyalkylcarbonylamino, or alkoxyalkylaminocarbonyl group.

5. The composition as claimed in claim 4 wherein each R group comprises a triiodophenyl group attached via a 1 to 10 atom bridge composed of bridging atoms selected from O, N and C.

6. The composition as claimed in claim 1 wherein said derivative is iohexyl hexaacetate, iopamidol penta-acetate, methyl diatrizoate or dimethyl dipamidate.

7. The composition as claimed in claim 1 wherein said polymer is biodegradable.

8. The composition as claimed in claim 1 wherein said polymer is biocompatible.

9. The composition as claimed in claim 1 wherein said polymer comprises polylactic acid, polycaprolactone, polyglycolic acid, polylactide-co-glycolide.

10. The composition as claimed in claim 1 wherein said polymer comprises polyesters such as poly(L-lactide), poly(D,L-lactide), poly(caprolactone), poly(glycolic acid), poly(lactide-co-glycolide), poly(lactide-co-caprolactone), poly(glycolide-co-caprolactone), polytrimethylene carbonate, poly(3-hydroxybutyrate), poly(3-hydroxyvalerate), poly(4-hydroxybutyrate), poly(dioxanone) polyamides such as poly(caproamide), poly(hexamethylene adipamide), poly(p-phenyleneterephtalamide), polyhydrocarbones such as poly(ethylene), poly(propylene), poly(1-hexene), poly(1-hexene-co-4-methyl-1,4-hexadiene), poly(tetrafluoroethylene), poly(vinyl alcohol), polyacetals such as poly(formaldehyde), polyketals, polyglycols, polyurethanes, segmented polyurethanes, polyanhydrides, polyphosphazenes, polysulfones, silicones, ABS resins, natural polymers such as collagen, fibrin, polysaccharides such as chitosan.

11. The composition as claimed in claim 1 wherein said polymer is a homopolymer, block copolymer, random copolymer, graft copolymer or polymer blend.

12. The composition as claimed in claim 1 further comprising a medical agent.

13. The composition as claimed in claim 12 wherein said medical agent is selected from anti-proliferative agents (paclitaxel and the like), immunosuppressive agents (dexamethasone, rapamycin, tacromilus, mycophenolic acid and the like), anti-inflammatory agents (aspirin, ibuprofen, naproxen and the like) anti-matrix metalloproteinase, lipid lowering agents (simvastatin, lovastatin, pravastatin and the like), anti-thrombotic agents, antiplatelet agents (e.g. clopidogrel, ticlodipine, dipyridamole, epoprostenole, iloprostenole, argatroban and the like), antibiotics and antiseptics, e.g. gentamicin, colistin, erythromycin, clindamicin, penicillins, norfloxacin, chloramphenicol etc.

14. The composition as claimed in claim 12
wherein said medical agent is present in the form of a lipophilic ester.

15. A process for producing a radio-opaque composition as claimed in claim 1 wherein said process comprises adding said derivative of organoiodine contrast agent to said polymer and mixing.

16. The process as claimed in claim 15 wherein said polymers are in solution, bead or powder form.

17. The process as claimed in claim 15 wherein said derivative of a contrast agent and said polymer are heated to a melt under stirring.

18. Use of a radio-opaque composition as claimed in claim 1 in the manufacture of a radio-opaque article.

19. A radio-opaque article comprising a radio-opaque composition as claimed in claim 1.

20. An article coated with a radio-opaque composition as claimed in claim 1.

21. The article as claimed in claim 19 wherein said article is a medical device.

22. The article as claimed in claim 21 wherein said medical device is selected from catheters, tubes, strings, meshes, sutures, stents, cannulae, plugs, plates, rods, guide wires, shunts, screws, pins, prostheses, balloons, needles, clips, staples, scaffolds, drug delivery systems, endoprostheses of heart valves, endoprostheses of ligaments, tendons and muscles and dental filling composites.

23. The article as claimed in claim 19 wherein said article is a toy.

24. The article as claimed in claim 20 wherein said article is a medical device.

25. The article as claimed in claim 20 wherein said article is a toy.
